(19) United States
(12) Patent Application Publication
(10) Pub. No.: US 2008/0249555 A1
(43) Pub. Date: Oct. 9, 2008

(54) Puncturing System

(75) Inventor: Herbert Harttig, Neustadt (DE)

Correspondence Address: BAKER & DANIELS LLP / ROCHE, 300 NORTH MERIDIAN STREET, SUITE 2700, INDIANAPOLIS, IN, 46204, US

(73) Assignee: ROCHE DIAGNOSTICS OPERATIONS, INC., Indianapolis, IN (US)

(21) Appl. No.: 12/057,884

(22) Filed: Mar. 28, 2008

(30) Foreign Application Priority Data
Mar. 29, 2007 (EP) 07006519.8

(51) Int. Cl. A61B 17/14

(52) U.S. Cl. 606/182; 606/181

(57) ABSTRACT

A puncturing system for obtaining a body fluid sample includes a lancet that is accelerated by a puncturing drive for a puncturing motion in the direction of puncturing, whereby the puncturing system comprises an adjustment facility for adjusting the puncturing depth up to which a lancet penetrates into the body of a patient during a puncture. The adjustment facility includes a limit stop element that can be moved transverse to the direction of puncturing and comprises a first side that faces the body of a patient during a puncture and a second side having a limit stop surface against which a limit stop that is connected to the lancet hits during a puncture. The distance from the limit stop surface to the first side of the limit stop element with respect to the lancet changes as the limit step element is moved in a direction transverse to the direction of puncturing. Accordingly, movement of the limit stop element transverse to the direction of puncturing may be used to adjust the distance that the tip of a lancet projects beyond a first side of the limit stop element during a puncture.

### BACKGROUND

1. Field of the Invention

The invention refers to a puncturing system for obtaining a body fluid sample.

2. Description of Related Art

Puncturing systems are used, for example, by diabetics, who need to check their blood sugar levels multiple times daily and for this purpose need a body fluid sample, usually blood or interstitial fluid, that is obtained from a puncturing wound that is generated using a puncturing system.

For the sample to be obtained with as little pain as possible, the puncturing depth up to which a lancet penetrates into the body of a patient during the puncturing should generally be kept to a minimum. Since the thicknesses of the upper layers of skin vary between patients and are also dependent on the body part selected for obtaining the sample, puncturing systems usually comprise an adjustment facility for adjusting the puncturing depth.

### SUMMARY OF THE INVENTION

It is the object of the invention to devise a way of creating a reliable able adjustment facility for a puncturing system for adjusting the puncturing depth.

In an adjustment facility according to embodiments of the invention, the puncturing depth is adjusted by means of a limit stop element that can be moved transverse to the direction of puncturing. The limit stop element has a bottom side that faces the body of a patient during a puncture and a top side having a limit stop surface against which a limit stop that is connected to the lancet hits during a puncture.

An adjustment facility according to embodiments of the invention includes of a wedge-shaped limit stop element. By shifting the limit stop element transverse to the direction of puncturing, it can be defined which site of the limit stop surface is hit during a puncture by the limit stop that is connected to the lancet. Since the extension of the limit stop element in the direction of puncturing, from the limit stop surface to the bottom side, changes transverse to the direction of puncturing, transverse shifting of the limit stop element can be used to define how far the tip of a lancet projects beyond the bottom side of the limit stop element during a puncture, and thus the puncturing depth can be defined. In the process, there is no need to provide that the limit stop element can be shifted exactly and only transverse to the direction of puncturing. Rather, in embodiments, the limit stop element for adjusting the puncturing depth may be moved in a direction that also has a component that is parallel to the direction of puncturing.

In embodiments, the limit stop element moves in a direction mainly transverse to the direction of puncturing while adjusting the puncturing depth. Furthermore, the limit stop surface and the bottom side can be flat surfaces that extend at an oblique angle with respect to each other. Accordingly, a change of the puncturing depth is proportional to a shift of the limit stop element transverse to the direction of puncturing. However, in embodiments, the limit stop surface may be configured in the form of a curved surface such that no linear proportionality exists between a shift of the limit stop element transverse to the direction of puncturing and a change of the puncturing depth.

In a puncturing system according to embodiments of the invention, there is no need for the body part in which a puncturing wound is to be generated to be touched to the bottom side of the limit stop element at the time the puncture is triggered. In embodiments, the limit stop element may be pressed against the body part only upon the limit stop of the lancet hitting the limit stop. This can be accomplished, for example, by the limit stop element being attached resiliently to the puncturing device or a lancet cartridge such that the limit stop surface can move in the direction of puncturing in a spring-like fashion when it is hit by the limit stop that is connected to a lancet. In embodiments, the limit stop element may be arranged in a rigid fashion such that a lancet is stopped as soon as the limit stop that is attached to it hits the limit stop surface.

The limit stop that is connected to the lancet and works in conjunction with the limit stop element in order to limit the puncturing depth can be provided, for example, in the form of a shoulder of a lancet. It should be noted that in embodiments, the flat lancet may be cut from a sheet of metal. In embodiments, the limit stop may comprise a lancet body made of plastic that bears a lancet tip made of metal, ceramics or any other sufficiently hard material. In embodiments, the limit stop need not be permanently connected to the lancet. In embodiments, the limit stop may be connected to the lancet during a puncture such that stopping the limit stop also effects ensuing stopping of the lancet. For example, the limit stop can be part of the lancet drive that couples to the lancet for the purpose of a puncture and releases the lancet thereafter.

A puncturing system according to embodiments of the invention can comprise a puncturing device and replaceable lancet cartridges that can be inserted into the puncturing device and each contain multiple lancets. In this context, the limit stop element can be part of the puncturing device or can be contained in a lancet cartridge of this type.

### BRIEF DESCRIPTION OF THE DRAWINGS

Further details are illustrated in the exemplary embodiments described below making reference to the appended drawings.

FIG. 1 shows an exemplary embodiment of a puncturing device of a puncturing system;

FIG. 2 shows an internal view of what is shown in FIG. 1; and

FIG. 3 shows a top view of the limit stop element of the puncturing system shown.

Although the drawings represent embodiments of various features and components according to the present invention, the drawings are not necessarily to scale and certain features may be exaggerated in order to better illustrate and explain the present invention. The exemplification set out herein illustrates an embodiment of the invention, and such exemplification is not to be construed as limiting the scope of the invention in any manner.

### DETAILED DESCRIPTION

For the purposes of promoting an understanding of the principles of the invention, reference will now be made to the embodiment illustrated in the drawings, which is described below. It will nevertheless be understood that no limitation of the scope of the invention is thereby intended. The invention includes any alterations and further modifications in the illustrated device and described method and further applications of the principles of the invention, which would normally occur to one skilled in the art to which the invention relates. Moreover, the embodiment was selected for description to enable one of ordinary skill in the art to practice the invention.

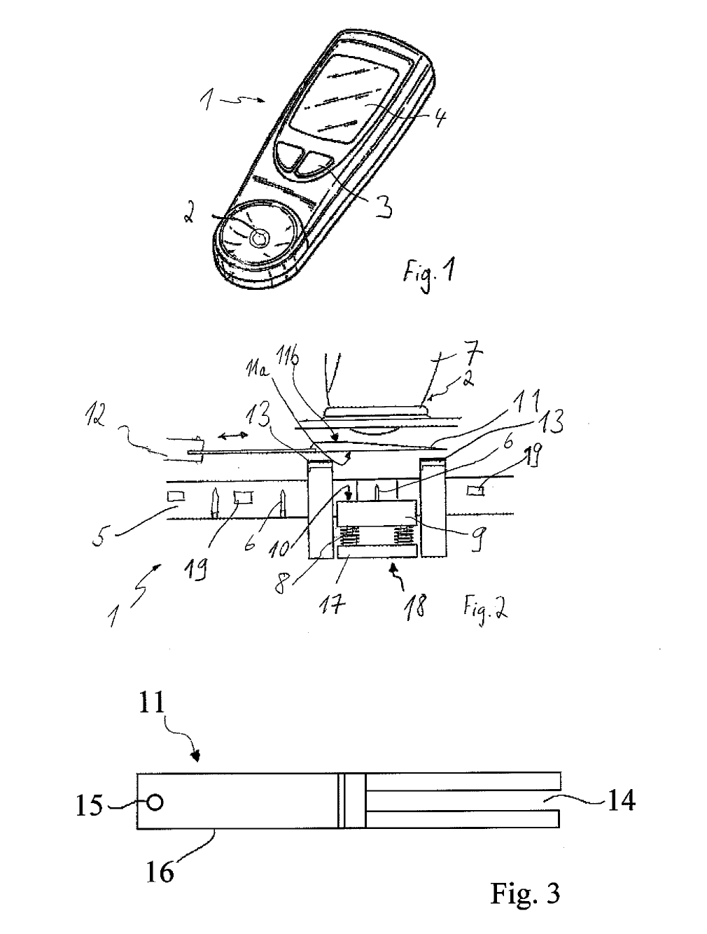

FIG. 1 shows a schematic view of an exemplary embodiment of a puncturing device 1 which, jointly with replaceable lancets, forms a puncturing system for obtaining a body fluid sample. The puncturing device 1 comprises a device opening 2 against which a body part is pressed in order to generate a puncturing wound. In the embodiment shown, the puncturing device has operating elements 3 in the form of buttons and a display facility 4 in the form of a liquid crystal display for displaying device setting and test results that were obtained from body fluid samples using a measuring facility (not shown) that is integrated into the puncturing device 1. The puncturing device 1 has a receptacle compartment (not shown) for a lancet carrier 5 having multiple lancets 6 that is shown in FIG. 2. The receptacle compartment has an opening that can be closed and is situated on the back side of the exemplary embodiment shown in FIG. 1.

In an internal view of what is shown in FIG. 1, FIG. 2 schematically shows certain components of the puncturing system 1 in the area of the housing opening 2 against which a body part 7, such as a finger for example, is touched in order to obtain a body fluid sample. In order to generate a puncturing wound, the puncturing drive 18 accelerates a lancet 6 for a puncturing motion in the direction of puncturing.

In the exemplary embodiment shown, the lancets 6 are arranged on a belt-shaped carrier 5. In order to convey the lancets 6 one after the other into a puncturing position, in which they can be accelerated in the direction of puncturing by the puncturing drive 18 for a puncturing motion, a stepping mechanism is provided, but not shown here. The stepping mechanism can, for example, comprise a reeling facility that can be used to reel the belt-shaped lancet carrier 5, which can for example be inserted into the receptacle compartment of the puncturing device 1 in a reeled fashion or folded into a stack in a zig-zagging fashion, onto a reel of the reeling facility and thus can be moved in a longitudinal direction. The belt-shaped lancet carrier 5 can, in embodiments, be contained in a replaceable cartridge, in which it is reeled much like the tape in a tape cassette. In a cartridge of this type, sections of the lancet carrier 5 with unused lancets 6 are reeled onto a first reel, whereby sections with used lancets 6 are reeled onto a second reel that is driven by the stepping mechanism such that the carrier 5 can be unreeled from the first reel and then reeled onto the second reel and, in the process, a fresh lancet 6 can be conveyed to the puncturing position by actuating the stepping mechanism.

As shown in FIG. 2, the lancets 6 are arranged on the belt-shaped lancet carrier 5 transverse to its longitudinal direction. A test field 19 for testing a body fluid sample that is obtained from a puncturing wound is arranged between each two lancets 6. In embodiments, the test fields 19 can contain test chemicals effecting a concentration-dependent change of optical properties for optical, in particular photometric, determination of an analyte concentration. In other embodiments, the test fields 19 may be configured for an electrochemical or physical test of a body fluid sample.

The puncturing drive 18 of the exemplary embodiment shown couples to the lancet carrier 5 such that the latter is moved in the direction of puncturing during a puncture jointly with a lancet 6 that is positioned in puncturing position. For this purpose, the puncturing drive 18 comprises a receptacle 9 for the lancet carrier 5, for example in the form of a groove, through which the lancet carrier 5 is guided. The receptacle 9 engages the lancet carrier 5 during a puncture in a clamping fashion and releases it after a puncturing and a returning motion are completed. In this manner, the lancet carrier 5 can be moved in a longitudinal direction through the receptacle 9 by the stepping mechanism, while a relative motion with respect to the receptacle 9 in the direction of puncturing is prevented.

In the exemplary embodiment shown, the receptacle 9 of the puncturing drive 18 forms a limit stop 10 that is connected to a lancet 6 that is positioned in puncturing position. For control of the puncturing depth, the limit stop 10 formed by the receptacle 9 cooperates with the limit stop element 11 of an adjustment facility 12 for adjusting the puncturing depth up to which a lancet 6 penetrates into the body of a patient during a puncture. The receptacle 9 is suspended via one or more compensating springs 8 on a brace 17 that is moved in the direction of puncturing during a puncture. When the puncturing depth is small, the compensating springs 8 compensate for the advancement carried out by the brace 17 such that the puncturing drive 18 does not get blocked. In the exemplary embodiment shown, the puncturing drive 18 is therefore coupled to the lancet 6 via compensating springs 8 that are being compressed when the lancet 6 is being stopped. The puncturing drive 18 can, for example, be a rotor drive, in which a connecting rod (not shown) effects the advancement motion of the brace 17. Rotor drives of this type are known from U.S. Pat. No. 7,223,276, the complete disclosure of which is hereby expressly incorporated herein by reference.

When a lancet 6 that is positioned in the puncturing position is moved jointly with the lancet carrier 5 in the direction of puncturing by the puncturing drive 18, a longitudinal edge of the lancet carrier 5 facing the device opening 2 first hits against a bending facility 13 that causes the lancet carrier 5 to bend in longitudinal direction such that the tip of the lancet 6 lifts off the lancet carrier 5. In the exemplary embodiment shown, the bending facility 13 is formed by two oblique surfaces that extend at an oblique angle with respect to the direction of puncturing and have the lancet 6 positioned between them in a puncturing position for a puncture.

During a puncturing motion, the limit stop 10 that is formed by the receptacle 9 of the puncturing drive 18 hits against a side 11a of the limit stop element 11 that is shown in a top view in FIG. 3. In the process, the side 11b of the limit stop element 11 is pressed by the puncturing drive 18 against a body part 7 that is being touched to the housing opening 2. The lancet 6 can therefore penetrate into the body part 7 during a puncture only as far as the tip of the lancet 6 projects beyond the side 11b when the limit stop 10 of receptacle 9 touches against the side 11a of the limit stop element 11.

As shown in FIG. 3, the limit stop element 11 has a slit 14 through which a lancet 6 moves during a puncture. By this means, the limit stop 10 that is formed by the receptacle 9 of the puncturing drive 18 can be stopped on both sides of the slit 14 by the limit stop element 11 such that tilting can be prevented. However, in principle it is sufficient if the limit stop element 11 interacts with the limit stop 10 on only one side of the lancet 6, for example in that the limit stop element 11 comprises an edge past which the lancet 6 is guided during a puncture and against which the limit stop 10 that is connected to the lancet 6 hits.

The limit stop element 11 is moveable transverse to the puncturing direction in the direction of the double arrow shown in FIG. 2. Since the extension of the limit stop element 11 in the puncturing direction from the side 11a forming the limit stop surface to the side 11b changes transverse to the puncturing direction, moving the limit stop element 11 transverse to the direction of puncturing allows to adjust how far the tip of a lancet 6 projects beyond the side 11b of the limit stop element 11 during a puncture. The limit stop side 11a and the side 11b of the limit stop element 11 extend at an oblique angle with respect to each other and each are level.

In the exemplary embodiment shown, the limit stop element 11 has a wedge-shaped section such that the thickness of the limit stop element 11 in a direction that is transverse to the puncturing direction changes. Therefore, shifting the limit stop element 11 in this direction that is transverse to the puncturing direction provides for adjustment of the puncturing depth in an easy fashion. The limit stop element 11 has a coupling element 15 for coupling to a shifting mechanism (not shown) of the adjustment facility 12. In the exemplary embodiment shown, the coupling element 15 is provided in the form of a hole. For coupling to the shifting mechanism, a peg (not shown) can engage the hole 15 and thus produce a form-fitting coupling in a known manner.

The limit stop element 11 shown has a section 16 that extends transverse to the puncturing direction and on which it is being held. The section 16 is adjacent to the wedge-shaped section of the limit stop element 11. The section 16 has a thickness that is smaller than the average thickness of the section forming the side 11a, this corresponds to the wedge-shaped section in the exemplary embodiment shown, and in embodiments, the section 16 may include a foil due to the low weight of the limit stop element 11. As explained above, the limit stop element 11 is moveable in the puncturing direction in a spring-like fashion. It should be noted that in embodiments, the limit stop element 11 may be rigid. During a puncture, it can therefore be pressed against a body part 7 by the limit stop 10 that is connected to the lancet 6. As soon as the lancet 6 is pulled back again by the puncturing drive 18, the limit stop element 11 springs back into its original position such that it is protected from contamination by body fluid that exits from the puncturing wound thus generated. In the exemplary embodiment shown, the side 11b of the limit stop element 11 therefore touches the body part 7 of the patient only for a part of the time during a puncture. However, in embodiments, the puncturing system may be configured to allow the side 11b of the limit stop element 11 to touch the body part 7 of a patient as soon as the body part 7 is placed against the housing opening 2.

In embodiments, the section 16 can also be attached on the thin end of the section forming the limit stop surface 11a or, in embodiments, on both ends.

In the exemplary embodiment shown, the thickness of the section 16 may be less than approximately 1 mm, in particular less than approximately 0.5 mm, namely a thickness of approximately 0.3 mm or less. The section forming the limit stop surface 11a differs in thickness between its ends by at least approximately 1 mm, in particular approximately 1.5 mm, such that the puncturing depth can be varied in the corresponding range. In the exemplary embodiment shown, the thickness of the wedge-shaped section of the limit stop element 11 varies between approximately 0 mm and approximately 1.5 mm. In embodiments, the thickness of the wedge-shaped section of the limit stop element 11 varies between approximately 0 mm and approximately 2 mm. The length of the limit stop surface 11a and thus the length of the slit 14 can be selected to be relatively large, i.e. in excess of approximately 1 cm or even in excess of approximately 2 cm, in order to minimize the influence of positioning inaccuracies of the limit stop element 11 on the adjusted puncturing depth. The width of the slit 14 can be, for example, between approximately 2 mm and approximately 4 mm.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A puncturing system for obtaining a fluid sample from a body of a patient including:
a lancet;
a stop connected to the lancet;
a puncturing drive for moving the lancet in a first direction;
an adjustment facility for adjusting the depth the lancet extends into the body, the adjustment facility comprising a limit stop element moveable in a direction transverse to the first direction, the limit stop comprising a first side that faces the body and a second side including a limit stop surface;
wherein the limit stop of the lancet contacts the limit stop surface when the lancet punctures the body; and
wherein movement of the limit stop element in the direction transverse to the first direction controls the depth the lancet extends into the body.

2. The puncturing system according to claim 1 wherein the first side extends at an oblique angle with respect to the limit stop surface.

3. The puncturing system according to claim 1 wherein the limit stop element includes a wedge-shaped section.

4. The puncturing system according to claim 1 wherein the limit stop element comprises a slit through which the lancet projects while puncturing the patient.

5. The puncturing system according to claim 1 wherein the limit stop element comprises a section that extends in a direction transverse to the first direction.

6. The puncturing system according to claim 5 wherein the limit stop element is moveable in the first direction.

7. The puncturing system according to claim 5 wherein the section is less than approximately 1 mm in thickness.

8. The puncturing system according to claim 7 wherein the section is less than approximately 0.5 mm in thickness.

9. The puncturing system according to claim 1 wherein the first side of the limit stop element touches the patient as the lancet punctures the patient.

10. The puncturing system according to claim 1 wherein the puncturing drive includes the limit stop.

11. The puncturing system according to claim 1 wherein the puncturing drive is coupled to a lancet during a puncture by a compensating spring.

12. The puncturing system according to claim 1 wherein the compensating spring compresses as the lancet is being stopped.

13. A puncturing system for obtaining a fluid sample from a body part of a patient comprising:
a lancet carrier including a lancet;
a receptacle connected to the lancet carrier and configured to receive a portion of the lancet carrier, the receptacle including a stop surface;
a drive configured to move the receptacle and including at least one spring;
a limit stop element including a slot, a first surface and a second surface located opposite the first surface, the first surface being arranged in a non-parallel orientation with respect to the second surface and the slot configured to receive at least a portion of the lancet; and
a receiving area configured to receive at least a portion of the body part and including a surface;
wherein the drive moves the lancet in a first direction toward the body part when activated, and the stop surface of the receptacle contacts the first surface of the limit stop element and moves the limit stop element into contact with the body part while the lancet punctures the body part; and
wherein the limit stop element moves in a second direction perpendicular to the first direction and the movement of the limit stop element determines the depth that the lancet punctures the body part.

14. The puncturing system as set forth in claim 13 further comprising an adjustment facility connected to the limit stop element configured to control the position of the limit stop element.

15. The puncturing system as set forth in claim 14 wherein the stop surface of the receptacle is orientated substantially parallel to the first surface of the limit stop element.

16. The puncturing system as set forth in claim 14 wherein the second surface of the limit stop element is arranged in a non-parallel orientation with respect to the stop surface of the receptacle.

17. The puncturing system as set forth in claim 13 wherein the lancet carrier further includes at least one test field.

18. The puncturing system as set forth in claim 13 further including a bending facility configured to bend a portion of the lancet carrier as the lancet is moved toward the body part.

19. The puncturing system as set forth in claim 13 wherein the receiving area includes an aperture through which a at least a portion of the body part extends.

20. The puncturing system as set forth in claim 13 wherein the body part is a finger.
